(19) United States
(12) Patent Application Publication
(10) Pub. No.: US 2018/0368935 A1
(43) Pub. Date: Dec. 27, 2018

(54) COLOSTOMY OR ILEOSTOMY BAG CLEANING SYSTEM

(71) Applicant: Lazarus Care International Limited, Chislehurst (GB)

(72) Inventor: Willem Daniel Van Der Spuy, Bloemfontein (ZA)

(21) Appl. No.: 16/075,206

(22) PCT Filed: Nov. 17, 2016
(86) PCT No.: PCT/ZA2016/050048

§ 371 Date: Aug. 3, 2018

(30) Foreign Application Priority Data
Feb. 3, 2016 (ZA) 2016/00767

(51) Int. Cl. A61B 90/70; A61F 5/445

(52) U.S. Cl. CPC A61B 90/70; A61F 5/4405; A61F 5/445

(57) ABSTRACT

A colostomy or ileostomy bag cleaning system including; (i) a support defining a longitudinal axis; (ii) a base located at or near a first axial end of the support; (iii) an inlet for, in use, receiving fluid under pressure; (iv) a ring with a flange that is, in use, connectable to a corresponding connecting ring on a colostomy or ileostomy bag; (v) a conduit that is in fluid communication with the inlet at one end of the conduit and extends to the ring, defining an outlet from the conduit within the ring; and (vi) a seal between the ring and the conduit.

### BACKGROUND

The present invention relates a colostomy or ileostomy bag cleaning system. More specifically, the present invention relates to a colostomy or ileostomy bag cleaning system that includes a stand for supporting the bag over an open toilet bowl.

Colostomy bag cleaning systems are known. For instance, U.S. Pat. No. 7,842,018 to Schena et al describes a system for cleaning digested waste from a colostomy bag while the bag is connected to the user.

However, users typically clean colostomy bags by filling the bag with water at a basin and emptying the bag into a toilet. This method is cumbersome and not altogether hygienic.

It is an object of the present invention to provide a simple and hygienic system for cleaning a colostomy or ileostomy bag while maintaining the bag suspended over a toilet bowl.

### SUMMARY OF THE INVENTION

According to a preferred embodiment of the present invention, a colostomy or ileostomy bag cleaning system includes:
a support defining a longitudinal axis,
a base located at or near a first axial end of the support;
an inlet for, in use, receiving fluid under pressure;
a ring with a flange that is, in use, connectable to a corresponding connecting ring on a colostomy or ileostomy bag;
a conduit that is in fluid communication with the inlet at one end of the conduit and extends to the ring, defining an outlet from the conduit within the ring; and a seal between the ring and the conduit.

Typically, the stand separates the ring from the base by between 50 cm and 80 cm, preferably by between 50 cm and 60 cm.

Generally, when the base is, in use, supported on a horizontal, level floor, the longitudinal axis of the support deviates from the vertical by no more than 20 degrees, preferably by no more than 10 degrees.

The system may further include a connector associated with the inlet for connecting a flexible hose to the inlet.

Preferably, a transverse arm extends from the support at or near the second axial end of the support.

Typically, the transverse arm extends from the support at an angle between 70 degrees and 110 degrees.

Generally, the transverse arm is between 7 cm and 18 cm in length, preferably between 7 cm and 14 cm in length.

Typically, the ring with flange is connected to the transverse arm at or near the free end of the transverse arm.

Generally, the conduit extends along the transverse arm. Preferably, the transverse arm is the conduit.

The system may further include a valve for regulating flow of fluid along the conduit.

Optionally, the system also includes a first reservoir for storing soap or detergent, which reservoir is in fluid communication with the conduit. Preferably, the content of the first reservoir is, in use, discharged into the conduit by the venture effect.

The system may also include a second reservoir that is charged by fluid emanating from the inlet, and that discharges fluid into the conduit. Preferably, the second reservoir is, in use, elevated above the conduit to provide fluid under pressure to the conduit.

Typically, the diameter of the ring is between 45 mm and 60 mm.

Generally, the support is right circular cylindrical.

### BRIEF DESCRIPTION OF THE DRAWINGS

The invention will now be described in more detail, by way of example only, with reference to the accompanying drawings in which:

FIG. 1 is an upper perspective view of a colostomy or ileostomy bag cleaning system according to a preferred embodiment of the invention; and

FIG. 2 is an upper perspective view of the system in FIG. 1, in use.

### DESCRIPTION OF THE INVENTION

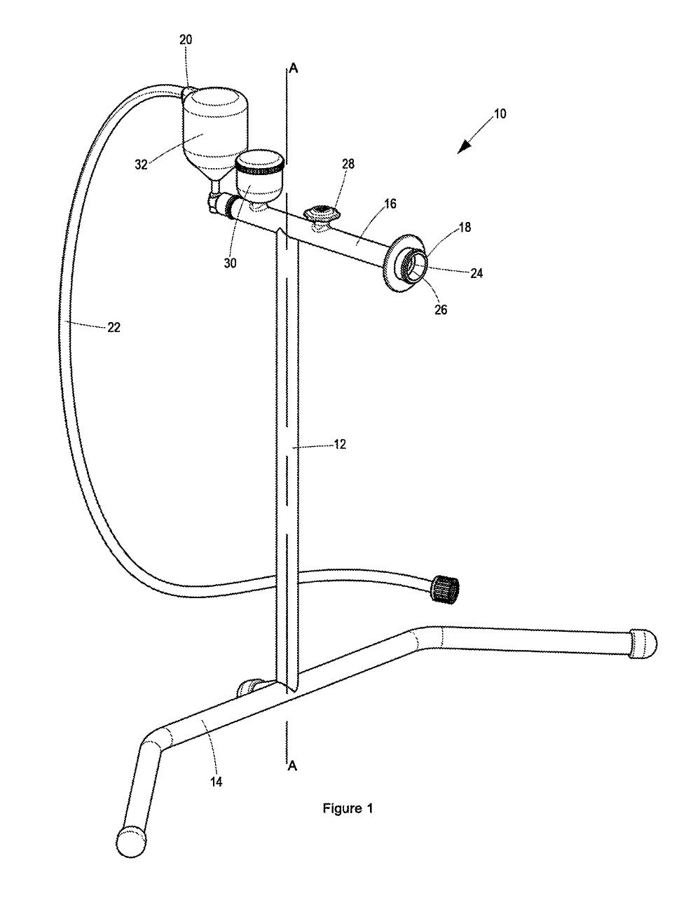

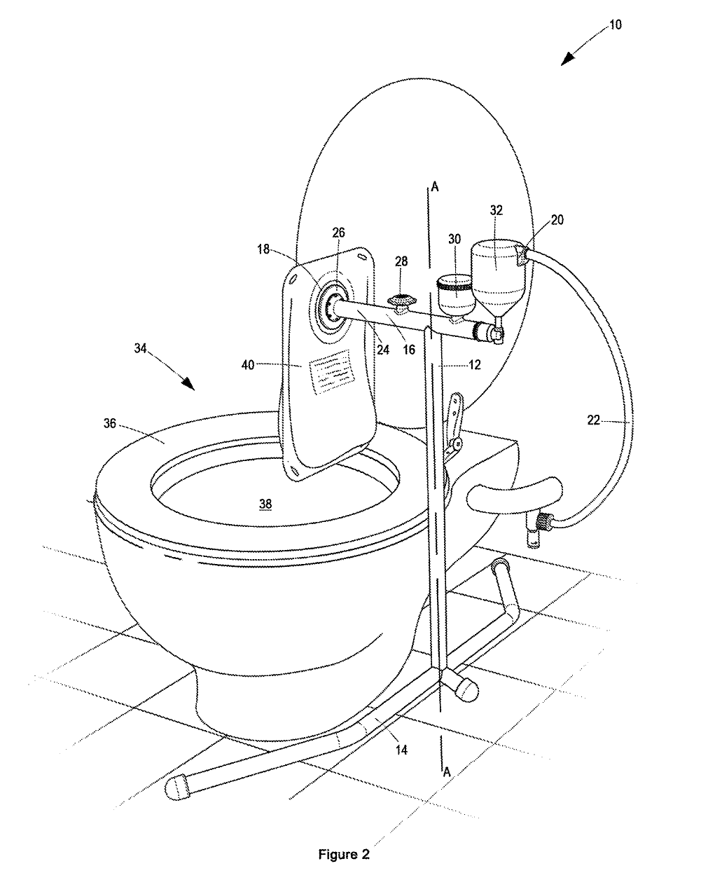

With reference to FIGS. 1 and 2, a colostomy or ileostomy bag cleaning system 10 includes a support 12 and base 14.

The support 12 is right circular cylindrical and defines a longitudinal axis A-A. It is between 50 cm and 80 cm, preferably between 50 cm and 60 cm in length, so as to extend above the standard height of a toilet seat.

The base 14 comprises three legs that extend from a first axial end of the support 12. When the legs 14 stand on a horizontal, level floor, the longitudinal axis of the support 12 is substantially vertical, i.e. angularly offset relative to the vertical by no more than 20 degrees, preferably by no more than 10 degrees.

Although the base 14 has been described as a set of legs that support the colostomy or ileostomy bag cleaning system 10 on the floor, it will be appreciated that the base could alternatively comprise a mount that is mountable to a wall. The mount could be a swivel mount, Furthermore, the mount could facilitate retraction/extension of the system 10 into/out of a wall cavity.

A transverse arm 16 extends from the second axial end of the support 12 at an angle between 70 degrees and 110 degrees. Preferably, the transverse arm 16 is orthogonal to the support 12 and is between 7 cm and 18 cm, preferably between 7 cm and 14 cm in length.

A ring with flange 18 (typically between 45 mm and 60 mm in diameter) is secured to the free end of the transverse arm 16 and is sized and shaped so as to be connectable to a corresponding connecting ring on a colostomy or ileostomy bag (such as a coloplast™ colostomy bag).

The system 10 also includes an inlet 20 with connector for connecting the inlet 20 to a flexible hose 22 that is, in turn, connected to a source of fluid under pressure (e.g. a water tap). In use, the inlet 20 receives fluid under pressure from the flexible hose 22.

Although the system 10 is described and illustrated as being fed with pressurised water from a tap via a flexible hose, it will be appreciated that the inlet could alternatively be fed with pressurised water or gas provided by a pump (e.g. a 12v pressure booster pump) or a compressed air canister. In such a configurations, the system 10 could be sold as a portable, self-contained system.

A conduit 24 is in fluid communication with the inlet 20 at one end of the conduit 24 and extends to the ring with flange 18, defining an outlet from the conduit 24 within the ring with flange 18. The Figures show the conduit 24 in the form of a pipe, which pipe also comprises the transverse arm 16. However, it will be appreciated that the conduit 24 could alternatively run alongside the transverse arm 16.

A seal 26 creates a seal between the ring with flange 18 and the conduit 24.

A valve 28 is located along the conduit 24 to regulate flow of fluid therein.

The system 10 further includes a first reservoir 30 for storing soap or a detergent, which first reservoir is in fluid communication with the conduit 24. The junction between the first reservoir 30 and conduit 24 is designed such that, when fluid is conveyed along the conduit 24, soap or detergent is withdrawn from the first reservoir 30 into the conduit 24 by the venture effect.

A second reservoir 32 is also in fluid communication with the conduit 24, upstream of the valve 28. The second reservoir 32 is, in use, charged with fluid emanating from the inlet 20. The second reservoir 32 is charged with fluid from a second reservoir inlet at or near the operative upper end of the second reservoir 32, and the fluid stored in the second reservoir 32 is not permitted to rise to the level of the second reservoir inlet. In use, the second reservoir 32 is also elevated above the conduit 24, so as to feed fluid under pressure into the conduit 24.

Turning specifically to FIG. 2, in use, the system 10 is positioned next to a toilet 34, with the transverse arm 16 extending over and past the toilet seat 36 so as to terminate over the toilet bowl 38. A colostomy or ileostomy bag 40 is secured to the ring with flange 18, and the flexible hose 22 is connected to a water tap. The second reservoir 32 is charged with water from the tap and the valve 28 is opened to permit fluid under pressure to be conveyed along the conduit 24 from the second reservoir 32, past the first reservoir 30 (from which some soap or detergent is withdrawn) and towards the outlet of the conduit 24. Since the outlet of the conduit 24 is located within the ring with flange 18, the fluid enters the bag 40, washing digested waste therefrom into the toilet bowl 38. Since the area between the ring with flange 18 and conduit 24 is sealed, all fluid exiting the conduit 24 flows into the bag 40, without leakage. When the bag 40 is clean, the valve 28 is closed, alternatively, the tap to which the flexible hose 22 is attached is closed and the bag 40 is disconnected from the ring with flange 18.

It will be appreciated that the system 10 could dispense with the first and second reservoirs 30 and 32. In such embodiment, the conduit 24 could extend directly between the inlet 20 and the ring with flange 18, with fluid pressure being provided directly by the water mains to which the flexible hose 22 is, in use, connected.

1. A colostomy or ileostomy bag cleaning system including:
a support defining a longitudinal axis;
a base located at or near a first axial end of the support;
an inlet for, in use, receiving fluid under pressure;
a ring with a flange that is, in use, connectable to a corresponding connecting ring on a colostomy or ileostomy bag;
a conduit that is in fluid communication with the inlet at one end of the conduit and extends to the ring, defining an outlet from the conduit within the ring; and
a seal between the ring and the conduit.

2. A system according to claim 1, wherein the stand separates the ring from the base by between 50 cm and 80 cm.

3. A system according to claim 1, wherein the stand separates the ring from the base by between 50 cm and 60 cm.

4. A system according to claim 3, wherein when the base is, in use, supported on a horizontal, level floor, the longitudinal axis of the support deviates from the vertical by no more than 20 degrees.

5. A system according to claim 3, wherein when the base is, in use, supported on a horizontal, level floor, the longitudinal axis of the support deviates from the vertical by no more than 10 degrees.

6. A system according to claim 5, wherein a connector is associated with the inlet for connecting a flexible hose to the inlet.

7. A system according to claim 6, wherein a transverse arm extends from the support at or near the second axial end of the support.

8. A system according to claim 7, wherein the transverse arm extends from the support at an angle between 70 degrees and 110 degrees.

9. A system according to claim 8, wherein the transverse arm is between 7 cm and 18 cm in length.

10. A system according to claim 8, wherein the transverse arm is between 7 cm and 14 cm in length.

11. A system according to claim 10, wherein the ring with flange is connected to the transverse arm at or near the free end of the transverse arm.

12. A system according to claim 11, wherein the conduit extends along the transverse arm.

13. A system according to claim 12, wherein the transverse arm is the conduit.

14. A system according to claim 13, further including a valve for regulating flow of fluid along the conduit.

15. A system according to claim 14, further including a first reservoir for storing soap or detergent, which reservoir is in fluid communication with the conduit.

16. A system according to claim 15, wherein the content of the first reservoir is, in use, discharged into the conduit by the venture effect.

17. A system according to claim 16, further including a second reservoir that is charged by fluid emanating from the inlet, and that discharges fluid into the conduit.

18. A system according to claim 17, wherein the second reservoir is, in use, elevated above the conduit to provide fluid under pressure to the conduit.

19. A system according to claim 18, wherein the diameter of the ring is between 45 mm and 60 mm.

20. A system according to claim 19, wherein the support is right circular cylindrical.
